SEQUENCE LISTING (1 sequence)

SEQ ID NO: 1 - DNA, 732 bp, Synthetic construct
  source 1..732: mol_type = other DNA; note = Example Dronpa Sequence
ggaggtggctccgggggtggtagtggcggtggctcaggctcggtcatcaagccggatatgaaaattaagttacgtatggaaggagcggttaatggtcatccgtttgctattgaaggcgtaggcttaggcaagccctttgaaggcaaacaaagtattgacttgaaagttaaggagggcgggccgttgccattcgcatacgacattctgacaacggccttctgctatgggaatcgcgtttttgcaaaatacccagagaacattgtagactactttaagcagtcgttcccagagggttactcgtgggaacgttcgatgaactacgaggatggcggtatctgtaatgccaccaacgatattacgttagatggcgattgttacatttacgagatccgctttcgcggcacaaattttccggcgaacggacccgtcatgcagaagcgcactgtaaagtgggagccttcaacagaaaatttatacgtccgtgacggcgttctgaaaggtgatgtcatcatggctctttctttggagggtggtggtcactaccgttgtgactttaaaaccacttataaggcaaagaaagtcgttcaattgccggactatcatttcgtagatcaccatattgagatcaaatcccacgataaagactattccaacgtgaatcttcacgaacatgcggaagcacattcggagcttccgcgtcaggccaaacaccaccatcaccatcattaataa

(19) United States
(12) Patent Application Publication
(10) Pub. No.: US 2026/0071962 A1
(43) Pub. Date: Mar. 12, 2026

(54) BIOSENSING OF RECONFIGURABLE ANALYTES USING INTEGRATED NANOPHOTONICS (BRAIN)

(71) Applicant: RTX BBN Technologies, Inc., Cambridge, MA (US)

(72) Inventors: Milica Notaros, Arlington, MA (US); Jacob A. Hammond, Boston, MA (US); Bryan A. Bartley, Friday Harbor, WA (US); Miles Thomas Rogers, Mansfield, MA (US); Michael Grace, Watertown, MA (US)

(21) Appl. No.: 19/236,123

(22) Filed: Jun. 12, 2025

Related U.S. Application Data
(60) Provisional application No. 63/659,051, filed on Jun. 12, 2024.
(60) Provisional application No. 63/696,504, filed on Sep. 19, 2024.

(51) Int. Cl. G01N 21/64; G01N 21/77; G01N 33/68

(52) U.S. Cl. CPC G01N 21/648; G01N 21/77; G01N 33/68; G01N 2021/7763

(57) ABSTRACT

Disclosed are embodiments photonic integrated circuits with photo-switchable components. Embodiments are illustrated in the form evanescent field perturbation sensors in which receptor molecules are patterned onto the sensor substrate with precision. In particular, light patterning and molecular-based photo-responsive motifs allow for the positioning of receptor molecules on the operational elements of the evanescent field perturbation sensors. Receptor molecules are further detachable from the sensor and capable of replacement with additional receptor molecules of the same type, or one or more alternative receptor molecules capable of receiving the same or different analyte receptor molecules. In certain embodiments the light sources for patterning and receptor molecule interchange may integrated within the sensor and/or sensor platform itself. In certain embodiments a method of recharging and/or reconfiguring the receptor molecules, and a machine for carrying out the method is disclosed.

### CROSS-REFERENCE TO RELATED APPLICATIONS

This application claims the benefit of priority to U.S. Provisional Application No. 63/659,051 filed Jun. 12, 2024, and U.S. Provisional Application No. 63/696,504 filed Sep. 19, 2024, the disclosures of which are incorporated herein by reference in their entirety.

### SEQUENCE LISTING

The Instant Application contains a Sequence Listing which has been submitted electronically in XML format and is hereby incorporated by reference in its entirety. Said XML copy, created on Aug. 15, 2025, is named “RAY0699US3” and is 2,940 bytes in size.

### BACKGROUND

Embodiments of the disclosure pertain to the targeted integration of photo-switchable proteins onto a photonic integrated circuit. Additional embodiments disclose a multi-layered photonic integrated circuit chip combined with photo-switchable proteins. Another embodiment discloses a specialized apparatus and process to activate or refresh the photo-switchable proteins integrated with photonic integrated circuits. Further embodiments pertain to reconfigurable evanescent field sensors.

In electronics, the field of photonic integrated circuits (“PICs,” alternatively, integrated optical circuits) detect, generate, transport, and process light in a fashion similar to electrons in integrated circuits. Typically composed of two or more photo elements, photonic integrated circuits may be stand-alone or combined with electronic integrated circuits. PICs may form some or all of a larger device and can be made into waveguides, power splitters, optical amplifiers, optical modulators, etc., It is a challenge fundamental to the construction of PICs to place the constitutive elements of a PIC with precision. Placement is particularly difficult in those applications in which multiple different compositional materials must be integrated to form a working PIC or PIC element. There is a clear need for PIC technologies that allow for the precise construction of PICS or PIC elements; this is especially so where there is a desire for reusability and/or reconfigurability.

PICs are often found in the fields of fiber-optic communications, photonic computers, sensors, and biosensors. The ability to selectively detect and/or quantify compounds of interest in a sample of unknown composition is an underlying goal of the sensor field. In particular, the rapid detection and, if possible, quantization of targeted compounds, such as biomarkers, is invaluable for application in fields such as: medical diagnostics; exposure risk evaluation for first responders and field inspectors; site exposure monitoring; agricultural monitoring (e.g., health of livestock, presence of pathogens in a crop); and, industrial settings (e.g., monitoring worker exposures, presence of pollutants).

Thus, there is a clear demand for the development of sensors with high selectivity, reproducibility, stability, sensitivity, and resolution. Selectivity describes the ability of a sensor to detect a target analyte in a sample containing other admixtures. Reproducibility is the ability to generate identical responses for repetitive experimental setups, which provides high reliability and robustness for an output signal. Finally, stability refers to the degree of susceptibility to ambient disturbances around the sensing system, which can affect the precision and accuracy of a sensor.

One technology developed in response to the demand relies on evanescent field perturbation sensing techniques including: interferometric, microcavity, photonic crystals, and Bragg grating waveguide-based sensors. Often, the techniques are embodied by silicon-based sensor platforms containing photonic integrated circuits. In broad terms, the sensors operate based on a sensitivity to changes in the local refractive index within the evanescent field surrounding the device. Affinity interactions between analyte and receptor molecules alter evanescent field properties generating a detectable signal. Evanescent field biosensors based on surface plasmon resonance or using planar waveguides have shown promise in the creation of sensitive, real-time, and label-free biomolecular detection sensors.

Despite the diversity of photonic integrated circuit fabrication techniques for the creation of sensor components such as waveguides, sample mixture channels, photon sources, etc. there is a deficiency in the optimal placement of receptor molecules. Techniques employing standard “wash” principles coat an entire substrate surface with receptor molecules without regard to any substructures that may be present within a given substrate. These substructures can include discrete integrated photonic circuit components such as underlying waveguides or evanescent field structures positioned at specific locations in the substrate. This lack of selectivity ultimately places receptor molecules in areas that do not contribute to the sensing function of the photonic integrated circuit which wastes resources and increases noise in the overall system. Thus, there is a clear need for a means to precisely combine receptor molecules into photonic integrated circuits.

Further, current techniques often allow only a single use of a sensor as the receptor molecule may be permanently bound to the underlying substrate and/or an analyte molecule bound to the receptor molecule may inactivate the receptor molecule, preventing further use. This leads to wastage of materials and increased expense for sensor operation and deployment. Also, a permanently bound receptor or analyte molecule configuration limits the ability of the underlying sensor platform to be reconfigured in the face of altered conditions to detect different targets of interest. Thus, there is a clear need for an evanescent field sensor with enhanced positioning of receptor molecules, with the ability to rapidly recharge for reuse, and with the ability reconfigure for the detection of different targets from an initial target.

### SUMMARY

Disclosed are systems and methods that can improve the state of the art in sensing by bringing gold-standard photonic and molecular detection capabilities and integrating them for use out of the lab by creating a small form factor, low size, weight, and power device that produces an immediate signal resulting from light-matter detection events by interaction of analytes (such as antibodies, aptamers, or other organic compounds) with integrated photonic components. In embodiments, photo-switchable proteins attach and detach different receptors to the surface of photonic integrated circuit components. Thus, embodiments of the disclosure encompass sensors that can be reprogrammed to detect new targets without requiring complex manufacturing or chemical procedures. In other embodiments, the sensor may be refreshed by detachment of spent receptors and replacement with fresh receptors.

In various embodiments, both antibodies and aptamers alone or in combination may be utilized. The best modality can be determined based on testing after utilizing the disclosure herein to determine the best sensing modality for the device, and the required stoichiometry for detection of different size particles can be determined through routine experimentation. For example, larger analytes such as bacterial cells or viruses may require fewer analyte molecules for detection, while smaller targets such as metabolites may require higher number of analyte molecules to bind enough of the target to create a detectable resonance shift.

Certain embodiments may take the form of an apparatus. The apparatus may comprise a photonic integrated circuit with at least one element. At least one photo-switchable protein may be attached to at least one element of the photonic integrated circuit. In other embodiments of the apparatus the photonic integrated circuit is combined with an integrated circuit. In still other embodiments, the photonic integrated circuit is the whole or part of a: waveguide, power splitter, optical amplifier, optical modulator, sensor, or biosensor. Certain other embodiments provide that the photo-switchable protein is a dimer; and, that the dimerization is controllable via exposure to at least one light source. In still other embodiments, the photo-switchable protein dimer is Dronpa. In still other embodiments, a glutaraldehyde is attached to a Dronpa monomer.

In certain other embodiments, the apparatus is an evanescent field perturbation sensor that may comprise: at least one sensor element with a surface; at least a first class of base receptor molecules attached to the surface; and, at least a first class of secondary receptor molecule. In these embodiments, the secondary receptor molecules are attached or detached from the base receptor molecules in response to photons of one or more prescribed wavelengths. Embodiments may further comprise a light source configured to illuminate at least one sensor element. In certain embodiments, the surface is a sample flow area. In still other embodiments, the base receptor molecules are attached to the surface via silanization. Other embodiments may further comprise at least a second class of secondary receptor molecules. The first class and second class of secondary receptor molecules may be configured to interact with different targets. The apparatus may further comprise at least a second class of secondary receptor molecules. The first class and second class of secondary receptor molecules may be configured to interact with different components of the same target.

Certain embodiments of the disclosure provide for a method. The method may comprise the steps of: providing at least one light source; providing a solution with analyte molecules; providing a photonic integrated circuit element with a surface; attaching to the surface receptor molecules; flowing the solution with analyte molecules over the surface of the photonic integrated circuit element; engaging the light source and binding the analyte molecules to the receptor molecules. In certain embodiments, the receptor molecules are attached via silanization. In still other embodiments, the surface is part of a sample flow area. In further embodiments, the analyte molecules are at least one selected from the group of: antibodies, antigens, enzymes, nucleic acids, a cell, a cellular structures, and a polymer. In certain embodiments, the method may further comprise the steps of: using the photonic integrated circuit element; and, recharging the photonic integrated circuit element after use. In embodiments, recharging the photonic integrated circuit element reconfigures the element. In still other embodiments, the method is performed with the apparatus herein described and/or claimed. In still other embodiments, an apparatus is provided and configured to perform the steps of the methods herein described and/or claimed.

In certain embodiments there is disclosed a method of reusing or reconfiguring a photonic integrated circuit. Embodiments of the method can comprise the steps: providing at least one photonic integrated circuit with at least one used element; configuring a lighting pattern to interact with the at least one used element; exposing the at least one used element to light in the lighting pattern, releasing secondary receptor molecules from base receptor molecules; flowing at least a first washing solution over the at least one used element, removing the released secondary receptor molecules creating a cleaned element; flowing at least a first recharging solution with new secondary receptor molecules over the cleaned element; exposing the new secondary receptor molecules and the cleaned element to light in the lighting pattern, binding the new secondary receptor molecules to the base receptor molecules; and, optionally repeating the above steps for one or more additional elements in series or in parallel.

### BRIEF DESCRIPTION OF THE SEVERAL VIEWS OF THE DRAWINGS

The accompanying drawings, which are incorporated into and form a part of the specification, illustrate several embodiments of the present disclosure; and, together with the description, explain the principles of the disclosure. The drawings are only for the purpose of illustrating an embodiment of the invention and are not to be construed as limiting the disclosure. In the accompanying drawings, like elements are numbered alike. Further objects, features and advantages of the disclosure will become apparent from the following detailed description taken in conjunction with the accompanying figures showing illustrative embodiments of the invention, in which:

FIG. 1 is a schematic representation of a basic evanescent field perturbation sensor array.

FIG. 2 presents a schematic of an individual sensor element from the sensor array of FIG. 1

FIG. 3 illustrates a first configuration where the base receptor molecule is made active by the addition of a secondary receptor molecule.

FIG. 4 represents the disassociation of the secondary receptor molecule from the base receptor molecule in response to a first wavelength of light.

FIG. 5 represents attachment of replacement secondary receptor molecules to the base receptor molecules in response to a second wavelength of light.

FIG. 6 shows the sensor element in a completed second configuration.

FIG. 7 schematically illustrates an embodiment of a sensor element in use.

FIG. 8 illustrates the domains of the Dronpa photo-switchable protein.

FIG. 9 presents a schematic illustration of an embodiment of the disclosure.

FIG. 10 illustrates in schematic a multi-level sensor platform embodiment.

FIG. 11 illustrates a method in accordance with embodiments of the disclosure.

FIG. 12, separated into figures FIG. 12A, FIG. 12B, and FIG. 12C, illustrates in schematic a machine in accordance with embodiments of the disclosure.

### DETAILED DESCRIPTION

A detailed description of one or more embodiments of the disclosed apparatus and method are presented herein by way of exemplification and not limitation with reference to the Figures. In certain embodiments, light patterning and molecular-based photo-responsive motifs allow for the positioning of receptor molecules at chosen positions as part of photonic integrated circuit devices or as complete photonic integrated circuits in and of themselves. Embodiments of the disclosure may also, optionally, be integrated with electron-based circuitry as part of a larger device. In certain embodiments, for example, a PIC may be integrated with digital CMOS (complementary metal oxide semiconductor) circuits configured to allow control and/or readout of the PIC.

In certain embodiments of the disclosure, a permanent base molecule is attached or localized to a photonic integrated circuit; or, to chosen elements within the photonic integrated circuit. The base molecule can bind to one or more different secondary molecules in a controlled spatial pattern that is reconfigurable and reusable. Thus, this targeted interaction with specific elements of a given photonic integrated circuit allow for the system to be imagined and configured or reconfigured for various applications. Practical applications can include usage in sensors, processors, network switches, etc.

Further disclosed are embodiments of evanescent field perturbation sensors in which receptor molecules are attached or localized onto a sensor element (e.g., a waveguide surface) with precision. In certain embodiments the attachment or localization may occur as the result of exposure to photonic emissions. In other embodiments, the localization or attachment may be the result of one or more differences in sensor material composition (e.g., sensor cladding material may be different in composition from a sensor element material). In certain embodiments the photonic integrated circuits make up the operational elements of evanescent field perturbation sensors. Receptor molecules are further detachable from the sensor and capable of replacement with additional receptor molecules of the same type, or one or more alternative receptor molecules capable of receiving the same or different analyte receptor molecules. In certain embodiments the light sources for patterning and receptor molecule interchange may be integrated within the sensor itself. In certain embodiments a method of recharging and/or reconfiguring the receptor molecules, and a machine for carrying out the method is disclosed.

The following detailed description is merely exemplary in nature and is not intended to limit the disclosure or the application and uses of the subject matter as described herein. Furthermore, there is no intention to be bound by any theory presented in the preceding background or the following detailed description. Theory and proposed mechanisms of action represent those as best understood at time of authorship but do not substitute or replace subsequent clarifications or amendments to the theory or mechanism.

As used herein the term “photonic integrated circuit” (PIC) refers to those circuits with optical functions. In practice a photonic integrated circuit contains two or more photonic components that form a functioning circuit. Photons are used to transfer, sense, process, and/or transmit information. Multiple components, or elements, may be integrated and fabricated on a single substrate to create the whole or part of a circuit. Components in the photonic integrated circuit can be both passive (splitters, multiplexers) and active (switches, detectors, lasers). Common materials for the construction of photonic integrated circuits include indium phosphide, silicon, silicon nitride, lithium niobate, silica, and gallium arsenide. Common techniques for construction include complementary metal-oxide-semiconductor (CMOS) or silicon-on-insulator (SOI).

As used herein the term “sensor” refers to a device which detects or measures a physical property and records, indicates, or otherwise responds to it. A sensor, in the broadest sense, produces an output signal for the purpose of detecting a physical phenomenon. Sensors may be composed of multiple components including a substrate, housing, electrical circuits, power source, etc. “Sensor elements” refers to one or more components of a sensor. For example, a sensor may be composed of the elements of a light source, waveguide, and detector with associated supporting hardware. In certain cases, “sensor element” or “sensing element” may also refer to that portion of the sensor which directly interacts with and/or responds to the physical event; in the immediate example above, the waveguide (as a whole, or with additional compositional features) may be viewed as the sensing element.

A “biosensor” is a type of sensor. A typical biosensor consists of three main parts: an interface functionalized with the bioreceptor where a specific biological event takes place, a transducer that converts a biological event into a measurable signal, and a detector that amplifies and processes the signal to be displayed. Bioreceptors can be immobilized on the interface surface either physically (i.e., adsorption, encapsulation, electrostatic, van der Waals, and hydrophobic interactions) or chemically (i.e., covalent bonding, and crosslinking). There are different types of bioreceptors available, and they can be generally classified into five major categories: (1) antibody/antigen, (2) enzymes, (3) nucleic acids (DNA, RNA), (4) cells and cellular structures, and (5) polymers.

As used herein the term “evanescent field sensor” is as used by those of skill in the art of optical sensors including those employing interferometer, microcavity, photonic crystal, and Bragg grating waveguides. “Evanescent fields,” in a common application, are characterized by exponential intensity decay, and are generated when light undergoes total internal reflection at a boundary between two substances of differing refractive index, for example, glass and air. Many evanescent field sensors are constructed using silicon as a waveguiding material using known techniques such as photolithographic etching, material doping, etc.

As used herein the term “light source” refers to an emitter of photonic energy. The light emitted by the source may be in one or more wavelengths and/or frequencies and may or may not be coherent. For example, a “light source” may refer to a laser emitted from a nanophotonic device. In still another example a “light source” may be as simple as the sun, a light bulb, a flame, or an LED.

As used herein the term “photo-switchable” or “photoreactive” refers to a property whereby an encounter with light at a single or multiple wavelengths induces a reaction. For example, two subunits of a protein complex may associate with each other at a first wavelength of light and disassociate with each other at a second wavelength while remaining stable or non-reactive when exposed to other wavelengths.

As used herein the term “receptor molecule” refers to a molecule interfaced with the photonic integrated circuit or elements thereof. For example, in the case of an evanescent field sensor, the receptor molecule is coupled to those sensor elements tied to the evanescent field such that interactions with the receptor molecule induces a detectable alteration in the evanescent field, leading to a sensor reading. Additionally, a given “receptor molecule” may have one or more additional components, or sub-structures, such as a “base receptor molecule” to which another “receptor molecule” may attach, forming a combination that may be referred to or viewed as a completed “receptor molecule.”

As used herein the term “analyte molecule” is one that interacts with the “receptor molecule” to induce a detectable change. The analyte molecule may be a target-of-interest within a sample mixture itself, or it may be one that binds to a target-of-interest and then interacts with the receptor molecule.

As used herein, particularly in the context of sensing, the terms “target” and “target of interest,” refer to a compound or composition desired for detection. By way of non-limiting examples, such a composition could include one or more small molecules or compounds, proteins, polymers, peptides, organic and inorganic compounds, etc. The target of interest may directly bind or otherwise interact with a receptor molecule or may bind or interact first with an analyte molecule which then binds or interacts with the receptor molecule.

A “peptide” or “protein” (used interchangeably) in the context of the present disclosure is to be understood as meaning a polymer composed of amino acids, preferably the 20 proteinogenic L-amino acids linked to one another via peptide bonds. In the context of this disclosure, the amino acids are given in a one-letter code, where, for example, C stands for cysteine, R for arginine, A for alanine and L for leucine. It is further understood that unless otherwise indicated, the amino acids in an amino acid sequence disclosed herein are linked via peptide bonds and, unless otherwise indicated, the sequence is listed in N- to C-terminal orientation. According to the disclosure, the peptide chains of the disclosure may be folded into individual proteins, protein subunits, or stand-alone peptide polymers.

Peptides and/or proteins can be chemically synthesized in various embodiments and/or recombinantly produced using protein design. Short peptides can easily be prepared synthetically, for example via solid phase synthesis. Longer peptides, polypeptides, and proteins, on the other hand, are often produced recombinantly in a host organism.

Typical acidic or negatively charged amino acids (depending on pH) are D and E.

The positively charged or basic amino acids (depending on the pH value) typically include R, K and H.

Amino acids such as G, A, C, I, L, M, F, V, P, S, T, W, Y, N and Q are typically uncharged, i.e., neutral, amino acids.

When reference is made herein to an “any” amino acid, what is commonly meant is one of the 20 naturally occurring proteinogenic amino acids, i.e. one of glycine (G), alanine (A), valine (V), leucine (L), isoleucine (I), Phenylalanine (F), Serine(S), Threonine (T), Proline (P), Methionine (M), Cysteine (C), Histidine (H), Lysine (K), Arginine (R), Glutamine (Q), asparagine (N), aspartic acid (D), glutamic acid (E), tyrosine (Y) and tryptophan (W). Unless otherwise stated, the amino acids are typically L-amino acids. In alternative embodiments, the peptide can also consist of D-amino acids, although it may be preferred that D- and L-amino acids do not occur at the same time within the peptides described herein.

The identity of nucleic acid or amino acid sequences is determined by sequence comparison. This sequence comparison is based on the BLAST algorithm established and commonly used in the prior art (cf. e.g. Altschul et al. (1990) Basic local alignment search tool, J. Mol. Biol., 215:403-410, and Altschul et al. (1997) Gapped BLAST and PSI-BLAST: a new generation of protein database search programs, Nucleic Acids Res., 25:3389-3402) and basically happens by similar sequences of nucleotides or amino acids in the nucleic acid or amino acid sequences be assigned. A tabular assignment of the relevant positions is called alignment. Another algorithm available in the art is the FASTA algorithm. Sequence comparisons (alignments), especially multiple sequence comparisons, are created using computer programs. For example, the Clustal series (see e.g. Chenna et al. (2003) Multiple sequence alignment with the Clustal series of programs, Nucleic Acid Res., 31:3497-3500), T-Coffee (see e.g. Notredame et al. (2000) T-Coffee: A novel method for multiple sequence alignments, J. Mol. Biol., 302:205-217) or programs based on these programs or algorithms. Sequence comparisons (alignments) are also possible using the computer program Vector NTI® Suite 10.3 (Invitrogen Corporation, 1600 Faraday Avenue, Carlsbad, California, USA) with the specified standard parameters, whose AlignX module for sequence comparisons is based on ClustalW, or Clone Manager 10 (Use of the BLOSUM 62 scoring matrix for sequence alignment at the amino acid level). Unless otherwise stated, sequence identity reported herein is determined using the BLAST algorithm.

Such a comparison also allows a statement to be made about the similarity of the compared sequences to one another. It is usually given as percent identity, i.e. the proportion of identical nucleotides or amino acid residues in the same positions or in positions corresponding to one another in an alignment. The broader concept of homology includes conserved amino acid exchanges in amino acid sequences, i.e. amino acids with similar chemical activity, since these usually exert similar chemical activities within the protein. Therefore, the similarity of the compared sequences can also be stated as percent homology or percent similarity. Identity and/or homology information can be made for entire polypeptides or genes or just for individual regions. Homologous or identical regions of different nucleic acid or amino acid sequences are therefore defined by matches in the sequences. Such areas often have identical functions. They can be small and contain only a few nucleotides or amino acids. Such small areas often perform essential functions for the overall activity of the protein. It can therefore make sense to relate sequence matches only to individual, possibly small areas. Unless otherwise stated, identity or homology information in the present application refers to the total length of the nucleic acid or amino acid sequence specified in each case.

Peptides and proteins according to the disclosure can have amino acid changes, in particular amino acid substitutions, insertions or deletions. Such peptides are further developed, for example, through targeted genetic modification, i.e., through mutagenesis processes, and optimized for specific purposes or with regard to special properties (e.g., in terms of their stability, binding, photoreactivity, etc.).

For example, targeted mutations such as substitutions, insertions or deletions can be introduced into the known molecules to change certain properties, for example. For this purpose, in particular a protein may be altered to contain a linker to a substrate. For example, the net charge of the peptides can be changed in order to influence substrate binding. Alternatively or additionally, one or more corresponding mutations can, for example, increase the stability or adsorption of the peptide. Advantageous properties of individual mutations, e.g., individual substitutions, can complement each other.

The term “conservative amino acid substitution” means the exchange (substitution) of an amino acid residue for another amino acid residue, whereby this exchange does not lead to a change in polarity or charge at the position of the exchanged amino acid, e.g., the exchange of a non-polar amino acid residue for another non-polar amino acid residue. Conservative amino acid substitutions within the scope of the disclosure include, for example: G=A=S, I=V=L=M, D=E, N=Q, K=R, Y=F, S=T, G=A=I=V=L=M=Y=F=W=P=S=T.

In preferred embodiments, a peptide or protein according to the disclosure can also be modified. Preferred modifications can be, for example, coupling the peptide with certain other molecules or chemical groups, for example organic (macro) molecules, for example via a covalent bond or a linker/spacer via a suitable amino acid of the chain and/or N- and/or C-terminal.

All the aforementioned features and embodiments disclosed herein can be implemented individually or in any combination.

Furthermore, a peptide according to the disclosure can also be at least one subunit (module) of a larger peptide or polypeptide, where the polypeptide can comprise a multimer of the sequences described herein.

The peptides described herein may have been chemically synthesized in various embodiments and/or recombinantly produced using protein design. Nowadays, short peptides can easily be prepared synthetically, for example using solid-phase synthesis such as Merrifield's solid-phase synthesis. Longer peptides and polypeptides, on the other hand, are often produced recombinantly in the host organism, e.g., in bacteria or yeast.

It is preferred to produce the proteins, peptides and/or peptide conjugates according to the disclosure using recombinant processes. This includes all genetic engineering or microbiological processes that are based on the genes for the peptides of interest being introduced into a host organism suitable for production and transcribed and translated by it (summarized in the context of this disclosure as biotechnological processes).

Using methods that are generally known today, such as chemical synthesis or the polymerase chain reaction (PCR) in conjunction with standard molecular biological and/or protein chemical methods, it is possible for a person skilled in the art to identify the corresponding nucleic acids and even complete genes using known DNA and/or amino acid sequences to produce. Such methods are, for example, from Sambrook, J., Fritsch, E. F. and Maniatis, T. 2001. Molecular cloning: a laboratory manual, 3rd Edition Cold Spring Laboratory Press. known.

As above noted, disclosed is an evanescent field sensor, in particular embodiments there is disclosed a silicon photonics-based system/method that utilizes photo-switchable receptor and analyte molecules to enable dynamic reconfiguration. In embodiments, the system utilizes light-activated proteins to reconfigure the sensor by optically releasing and attaching receptor molecules selective to different targets of interest via light from one or more light sources. This enables the ability to rapidly change analyte molecules and targets that can be detected.

As above discussed, one of the main challenges in creating customized and highly-selective sensors is the addition of components required to interface with the target and generate a signal for measurement (e.g., antibodies or aptamers in the case of some sensors) to the device itself. Prior methods are typically irreversible or require treatment with harsh chemicals and multistep protocols to attach new analyte or receptor molecules. The disclosed system advantageously overcomes this limitation by using a photo-switchable protein that dissociates when exposed to a specific wavelength of light and dimerizes when exposed to a second wavelength.

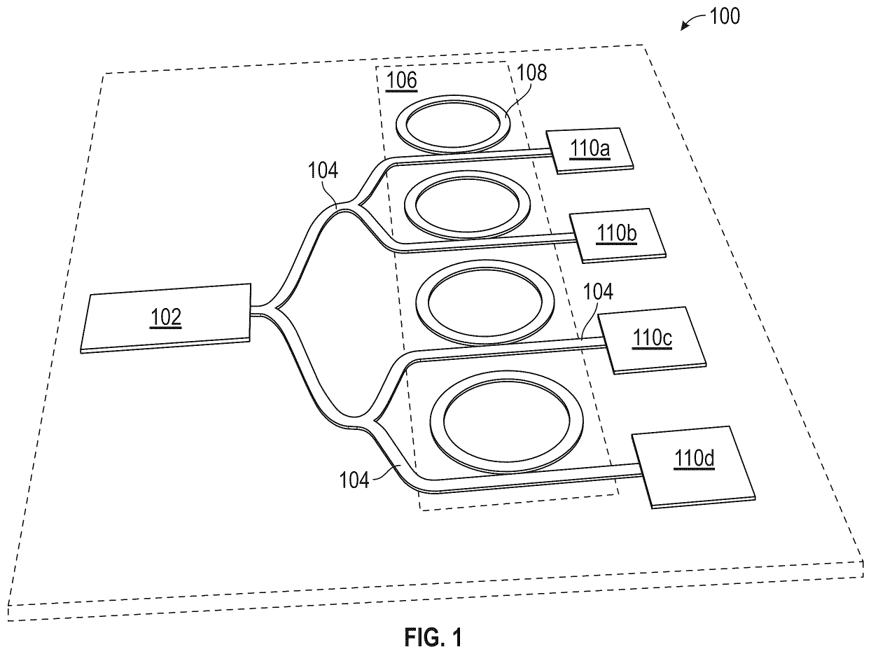

Presented in FIG. 1 is a schematic representation of an example photonic integrated circuit 100 for an evanescent field perturbation sensor array. The sensor waveguiding material is silicon such as is known in the art. In certain embodiments the waveguiding material is made of silicon which is clad with silicon dioxide (i.e., SiO₂). In certain embodiments, substrates in which the waveguiding material is found is dependent upon a wafer-scale lithography process. Common substrate materials can include lithium niobate (LiNbO₃) and silicon nitride (SiN).

A single light source 102 emits light in the form of a laser through a diverging-to-parallel set of four (4) waveguides 104 into a sample flow area 106. Within the sample flow area 106, the waveguides are configured as ring resonators 108. The waveguides 104 exit the sample flow area 106 into individual detectors 110 (shown as individuals a-d) which receive and transduce a signal indicating a sensor measurement. In operation, perturbations in the evanescent field in the ring resonators 108 generate the signal reported by the detectors 110. Thus, the ring resonators 108 serve as a sensor element 108 for the sensor. Thus, embodiments of the sensor also can comprise a light source, sensor element, and detector. Those of skill in the art can readily appreciate that the waveguides 104 may be singular from light source 102 to detector 1100, that more than four sensor elements are possible, and that each sensor element to detector connection may be viewed either as an individual “sensor” or, that the group of sensor elements in an array configuration may be referred to as a sensor. Although depicted in the form of a ring resonator, other forms, such as those herein listed, that make use of perturbations in an evanescent field are hereby taught and contemplated.

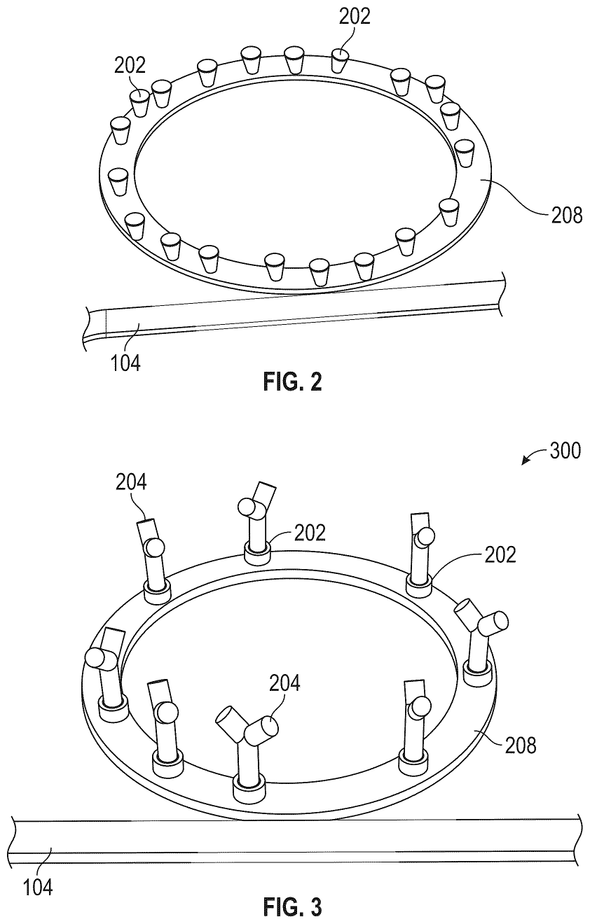

FIGS. 2-7 illustrate a schematic representation of an embodiment of the invention in the context of an individual ring-resonator style sensor element 208 from the array displayed in FIG. 1. In the illustrated embodiment, a secondary receptor molecule 204 is detached from a base receptor molecule 202 and replaced with a new secondary receptor molecule 206. One of skill in the art can readily envision that the same or variations of the below disclosed principles may be applied to one or more additional elements to form sensor element arrays capable of detecting one or more targets. In certain embodiments the secondary receptor molecule may, instead, be an analyte molecule.

Presented in FIG. 2 is a schematic of an individual ring-resonator style sensor element 208 in accord with the depictions in the sensor array of FIG. 1. A base receptor molecule 202 is first attached to the ring resonator sensor element 208. Advantageously, localization of the base receptor molecule 202 to the sensor element 208 increases signal while cutting down on noise and reducing wastage of receptor molecules. In certain embodiments, such as shown in FIG. 1, the ability to localize to a specific sensor element 208 allows configuration of a single platform chip with multiple sensor elements 208 to have each element 208 configured to respond to one or more additional targets. Or, in certain cases, sensor elements 208 may be configured to detect the same target, but with two different modes of action (e.g., targeting two different proteins found in the same virus; or, use of an antigen/antibody on one sensor element and DNA/RNA on a second sensor element). Those of skill in the art can appreciate that more than one type or class of base receptor molecule 202 may be attached to the ring resonator/sensor element 108/208. In certain embodiments the base receptor molecule 202 may be attached via the use of a patterned light source to selectively mask or expose sensor elements 208 to accomplish the localization. In certain embodiments, a protein, or protein subunit, serving as a base receptor molecule 202 may have a glutaraldehyde attached to it. The glutaraldehyde serves as an anchor molecule that links and binds the base receptor molecule 208 to the sensor element 208. In certain embodiments the anchor molecule is itself photoreactive to allow the selective placement and removal of the base receptor molecule 208. In still other embodiments, the base receptor molecules 202 may be applied as a “wash” across sample flow area 106 containing one or more sensor elements 208 with localization achieved by selective attachment of secondary receptor molecules 204 to the base receptor molecules 202 attached to sensor element 208.

Although depicted as containing a single type of base receptor molecule 202, one or more additional types or classes of base receptor molecule may be localized to the same sensor element 208. This allows, for example, the same sensor element to detect one or more additional targets of interest (e.g., a spike and envelope protein of a viral particle), or in the alternative, to detect the same target of interest with different mechanisms (e.g., via an antibody and a nucleic acid or peptide chain).

In still other embodiments, the surface of a sensor element 208 is silanized using an organosilane. In certain embodiments the organosliane is 3-aminopropyltriethoxysilane (APTES). In still other embodiments the organosilane may be 3-aminopropyltrimethoxysilane (APTMS), N-(2-aminoethyl)-3-aminopropyltriethoxysilane (AEAPTES), N-(2-aminoethyl)-3-aminopropyltrimethoxysilane (AEAPTMS), N-(6-aminohexyl) aminomethyltriethoxysilane (AHAMTES), 3-aminopropyldimethylethoxysilane (APDMES), 3-mercaptopropyltrimethoxysilane (MPTMS), glycidyloxypropyl-trimethoxysilane (GOPS), and others such as are known to those of skill in the art. Depending on the type of silane molecule used, the surface after the silane treatment can be aminated (e.g., APTES, APTMS, AEAPTES, AEAPTMS, and AHAMTES), thiolated (MPTMS), or epoxy-group functionalized (GOPS). Those of skill in the art will appreciate that those numerous techniques which can attach and/or bind the base receptor molecule to the sensor element without altering the functionality of the base receptor molecule readily fall within the scope of the principles and embodiments herein illustrated. In certain embodiments, only sensor elements 208 are silanized, thus allowing for localization of the base receptor molecules to the sensor elements.

FIG. 3 illustrates a first configuration 300 where a base receptor molecule 202 attached to a sensor element 208 is made active (“functionalized”) by the addition of a secondary receptor molecule 204. In this instance the secondary receptor molecule 204 is one that directly interacts with a target of interest, fulfilling the role of an analyte molecule. In operation, passage of an unknown mixture over the sensor element 208 results in the target of interest, if it is present in the unknown mixture, interacting with the secondary receptor molecule 204 inducing a change in the evanescent field of the sensor element 204. Passage of the unknown mixture may be accomplished by suspension or dissolution in a liquid or gas mixture, or by raw intake (e.g., atmospheric sampling).

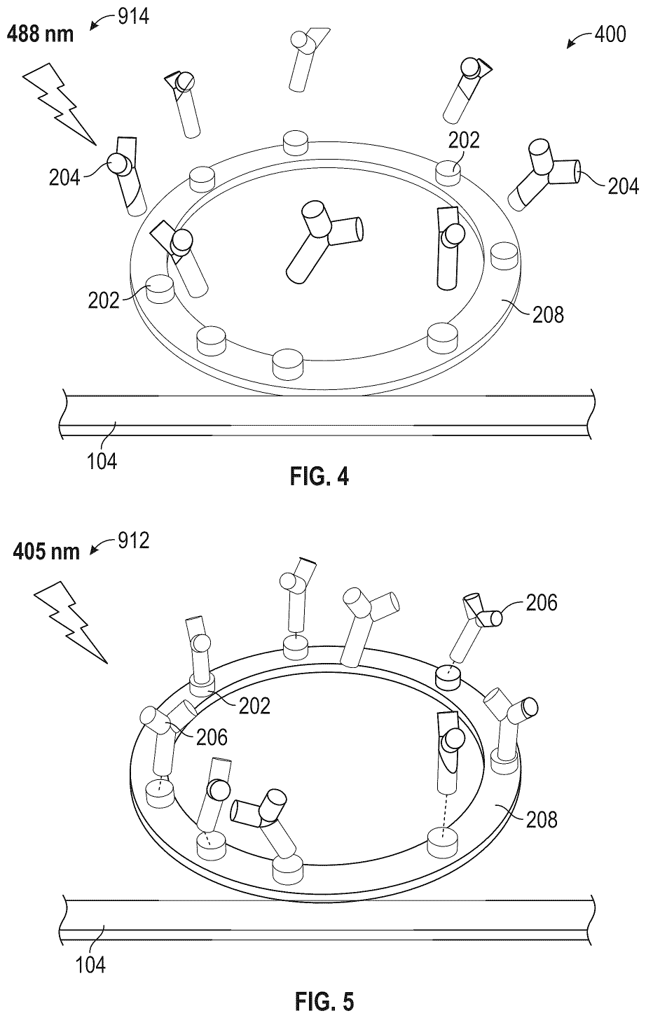

FIG. 4 schematically represents a transition configuration 400 in which the disassociation of the secondary receptor molecule 204 from the base receptor molecule 202 in response to a first wavelength of light. In this instance the light is at 488 nm, roughly termed “cyan” in color. The secondary receptor molecule 204 may be removed from the sensor element 208 and/or the sample flow area 106 via one or more washes with liquids or gases, via mechanical perturbation, via heating or vaporization, etc.

FIG. 5 represents attachment of replacement/new secondary receptor molecules 206 to the base receptor molecules 202. In this instance light at 405 nm, roughly termed “violet,” provides the energy to link replacement secondary receptor molecules 206 to the previously-occupied base receptor molecules. Although illustrated with light, those of skill in the art can readily appreciate that potentially any suitable secondary mechanisms may suffice to detach and replace secondary receptor molecules from the base receptor molecule. Such secondary mechanisms may be useful in situations where low or no power is available to provide light to clean and refresh the sensor platform. Secondary mechanism examples can include pH gradients, osmotic gradients, usage of solvents, acids and bases, heating and cooling, etc. Further, although cyan and violet are disclosed as the wavelengths of choice for the example, those of skill in the art can readily tailor embodiments of the invention to use the same or different wavelengths and to use light either alone or in combination with another technique, such as a pH gradient.

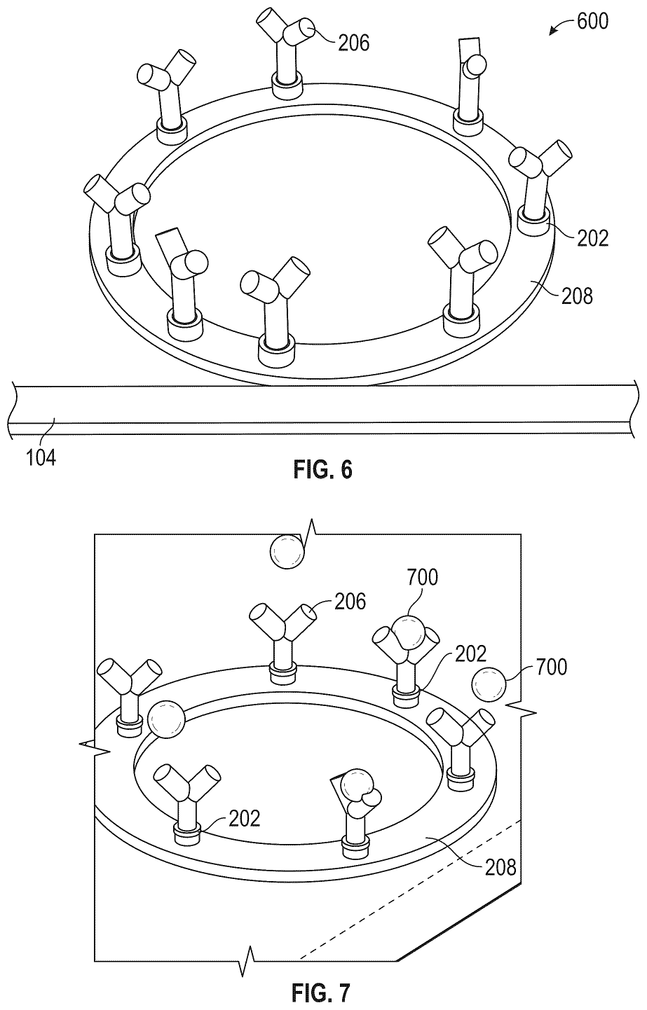

FIG. 6 shows the sensor element in a completed second configuration 600. Though the base receptor molecules 202 are original, refreshed and renewed secondary receptor molecules 206 are in place. The new secondary receptor molecules 206 may have the same, or a different target of interest from the original secondary receptor molecules 204. Thus, the sensor element 208 may be replenished for reuse, or reconfigured for use in a different use case scenario. The enablement of reuse or reconfiguration advantageously preserves the underlying sensor elements 108/208 and infrastructure thus lowering overall per-use cost, increasing sensor lifespan, and permitting rapid reconfiguration for optimization in the face of changing sensor environments and desired targets of interest.

Thus, the manufacture, use, and refreshment/reconfiguration process may be summarized as: First, as shown in FIG. 2, a base receptor molecule 202 is attached to a sensor element 208. Next, as shown in FIG. 3, secondary receptor molecules 204 are bound to the base receptor molecules 202; this may be done photonically. At this point the sensor element is ready for use (“functionalized”) and may be exposed to one or more sample mixtures. After use, as shown in FIG. 4 the ring resonator sensor element 108 is illuminated with light at a first wavelength (e.g., 488-nm cyan light), which releases the secondary receptor molecules 204 from the base receptor molecules 202. The sensor element 208 then undergoes a wash step to remove the secondary receptor molecules 204 and any other contaminants. In certain embodiments a solution containing the same or different secondary receptor molecules 206 is then provided to the ring resonator sensor element 108/208. Then, as illustrated in FIG. 5, the sensor element 208 is illuminated with a second wavelength (e.g., 405-nm violet light), which attaches the new secondary receptor molecules 206 to the previously-cleared base receptor molecules 202. Finally, as presented in FIG. 6, the reconfiguration process is complete, and the sensor element 208 is now activated with new secondary receptor molecules 206. In certain embodiments the new secondary receptor molecules 206 may be selective to a different target molecule 700 than the original secondary receptor molecules. Also, in certain embodiments, the secondary receptor molecules 204/206 may serve the role of analyte molecules directly interacting with the target of interest.

The same basic steps outlined above may be performed with elements of other photonic integrated circuit devices, allowing for those devices to also be reconfigured and/or refreshed. Example uses can include: Tunable nonlinearity, by leveraging photo-switchable proteins to add/remove material with nonlinear properties. Tunable modulators, by leveraging photo-switchable proteins to add/remove material to change the effective refractive index of an optical mode propagating in a waveguide. Tunable resonances, by leveraging photo-switchable proteins to add/remove material to tune the frequency resonance of a resonant device. Finally, example embodiments could be used to make up for fabrication variation if a device performance is slightly off from what was intended.

Example analyte molecules configured to a target of interest can include one or more antibodies, aptamers, artificially constructed peptides and proteins, polymers, etc. Those of skill in the art readily recognize such molecules as are capable of adaptation to the principles herein disclosed.

FIG. 7 schematically illustrates an embodiment of a sensor element 208 in use. Target analytes 700 exposed to the sensor element 208 bind to an analyte receptor molecule configured as a secondary receptor molecule 204/206 bound to a base receptor molecule 202 which is attached to the sensor element 208 surface.

Photo-switchable proteins are naturally occurring photoreceptor proteins that can serve as optical sensors or optical actuators. Certain members of the class, for example, modify transcriptional activity of the cell when exposed to light. Multiple photo-switchable proteins are identified and well characterized. Photo-switchable proteins are sourced from rhodopsins and are categorized as microbial (type I) opsins and animal (type II) opsins; the former type is found in microbes, such as archaea, cubacteria, fungi, and algae; and the latter is found in animals and humans. Microbial and animal rhodopsins share architectural similarities and both are composed of seven transmembrane α-helices, called opsins (also termed as apoproteins) and a photosensitive chromophore, called retinal. Upon illumination at a specific wavelength, retinal in microbial rhodopsins usually undergoes isomerization from all-trans to 13-cis that initiates a series of cyclic reactions, known as a photocycle, unique for each microbial rhodopsin type. This causes generation of unique photo-intermediates that induces conformational change of proteins, which causes channel opening, closing and ionic conductance of the rhodopsin. Example rhodopsins include: channelrhodopsin, halorodopsin, archaerhodopsin, and Leptosphaeria rhrodopsins. Within these protein classes are those that dimerize and/or otherwise associate and/or disassociate in response to the same or differing wavelengths of light.

Embodiments will utilize one such protein, Dronpa. This protein can be reliably toggled at 405 nm (on/dimer) and 488 nm (off/disassociated) (See FIGS. 5 and 6). This switching can occur over 50 times with no significant degradation. The binding strength of the two dimers in the on state is approximately 80 pN, able to withstand significant shear force in a microfluidic device.

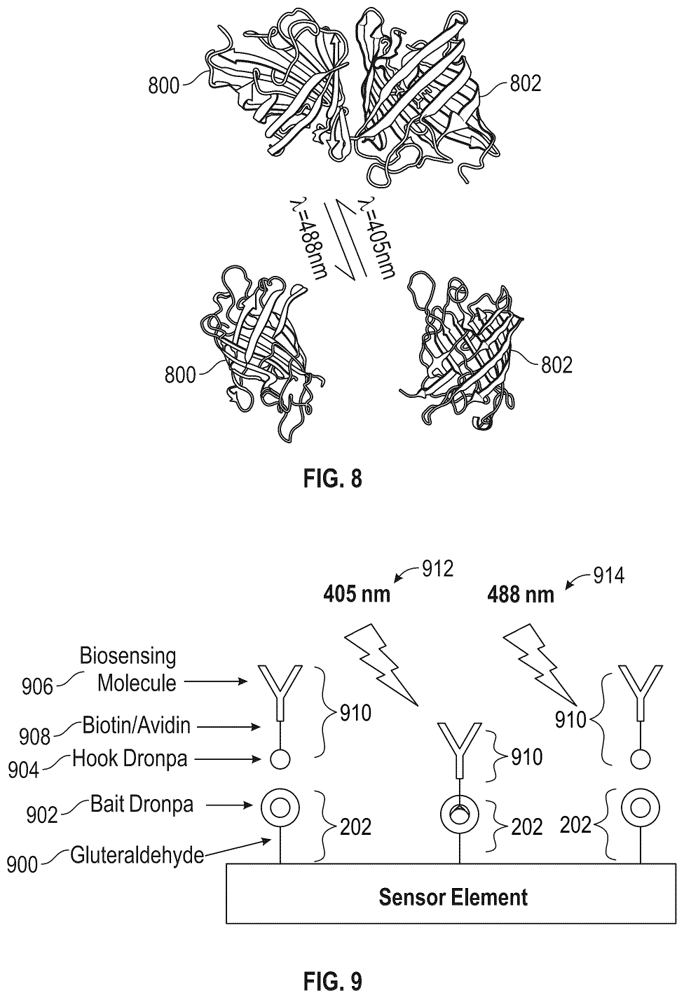

FIG. 8 illustrates the domains of the Dronpa photo-switchable protein. Dronpa is an engineered photochromic fluorescent protein from coral. The name “Dronpa” is composed of “Dron”, a ninja term for vanishing, and “pa”, which means photoactivation. Dronpa possesses two domains, a first domain 800 and a second domain 802, that dimerize when exposed to 405 nm. Embodiments herein may use one of these domains, a first domain 800, attached to the surface of the sensor element 108/208 as the base receptor molecule 202. In certain embodiments, the attachment of the first domain 800 to a silanized sensor element 208 surface is accomplished via a glutaraldehyde attached to the first domain.

The second domain 802 may then be attached (e.g., via biotin/streptavidin) to an analyte receptor creating a secondary receptor molecule 204/206. Upon photodimerization the first and second domains join creating an activated receptor capable of interacting with a target of interest and generating a signal. After usage, as above discussed, the secondary receptor molecule 204 can then be removed by exposing to 488 nm and washing. New secondary receptors 206 with the same, or different, analyte molecules may then be introduced and bound to the base receptor molecules.

Dronpa monomers may be expressed and purified using reported protocols. In certain embodiments Dronpa may be one with a sequence similarity of 70% or more to that of SEQ ID NO: 1 as below provided in Table 1.

TABLE 1
Example Dronpa Sequence-SEQ ID NO: 1

ggaggtggctccgggggtggtagtggcggtggctcaggctcggtc
atcaagccggatatgaaaattaagttacgtatggaaggagcggtt
aatggtcatccgtttgctattgaaggcgtaggcttaggcaagccc
tttgaaggcaaacaaagtattgacttgaaagttaaggagggcggg
ccgttgccattcgcatacgacattctgacaacggccttctgctat
gggaatcgcgtttttgcaaaatacccagagaacattgtagactac
tttaagcagtcgttcccagagggttactcgtgggaacgttcgatg
aactacgaggatggcggtatctgtaatgccaccaacgatattacg
ttagatggcgattgttacatttacgagatccgctttcgcggcaca
aattttccggcgaacggacccgtcatgcagaagcgcactgtaaag
tgggagccttcaacagaaaatttatacgtccgtgacggcgttctg
aaaggtgatgtcatcatggctctttctttggagggtggtggtcac
taccgttgtgactttaaaaccacttataaggcaaagaaagtcgtt
caattgccggactatcatttcgtagatcaccatattgagatcaaa
tcccacgataaagactattccaacgtgaatcttcacgaacatgcg
gaagcacattcggagcttccgcgtcaggccaaacaccaccatcac
catcattaataa

In certain embodiments the sensor waveguiding material is either silicon nitride or silicon. The silicon nitride or silicon surface may be configured to receive a Dronpa monomer serving as a base receptor molecule 202 by first modifying the surface through the process of silanization with (3-Aminopropyl)triethoxysilane (APTES) and washing. The Dronpa monomer may then be permanently bound to the surface though the use of glutaraldehyde 900. In certain embodiments, the silanization process enables attachment of the base receptor molecule 202 across the entire surface of a sensor area; for example, across the entire sample flow area 106 of FIG. 1. Secondary receptor molecules 204 may then be patterned onto individual sensor elements 208 or, alternatively, may also be bound to all the base receptor molecules 202. Alternatively, as the sensor element 208 material may be different from the material comprising the sensor body or substrate, differences in material composition may be used to guide attachment of one or more base receptor molecules 202 to the sensor element 208.

In order to establish a quantitative sensor that reports relevant amounts of the target analyte of interest, the relative resonant shift of the sensor when varying amounts of analyte are bound is established. Quantitation depends on many variables, a few include: the amount of target in the sample, the amount of base receptor molecules 202 bound to the surface of the sensor element 208, and the selectivity of the analyte molecules to the target of interest. The amount of receptor molecules and the selectivity of the analyte molecules directly contribute to the overall sensitivity of the sensor element 208. Additional factors contributing to sensor/sensor element sensitivity can also include: material composition, physical layout, light propagation properties, attenuation, system noise, light source wavelength, light source properties (e.g., purity of wavelength); and, light detector properties and/or sensitivity.

The disclosed photo-switchable attachment chemistry significantly cases the process of testing to find the relevant linear range for each target molecule. Relevant concentrations of the target molecule in a solution can be easily controlled through simple dilution. The amount of receptor molecules on the sensor surface can similarly be controlled by delivering different concentrations of receptor molecules to the device before attachment.

In certain embodiments varying amounts of bioreceptor molecules may be attached to the surface of the device to determine the linear range of detection at relevant concentrations. Such calibration methods are routine. By way of example a practitioner may perform 10-fold dilutions to discover a relevant concentration, then 2-fold dilutions to narrow down and discover the precise concentration of receptor molecules that must be attached to the sensor element surface to deliver quantitative information on the concentration of the target molecule in a solution. The process can be automated so that it can be performed on every target analyte the sensor will detect.

FIG. 9 presents a schematic illustration of an embodiment of the disclosure. At left: The device surface supports a first Dronpa monomer 902, termed a “Bait Dronpa,” covalently attached to the surface via glutaraldehyde 900 as a base receptor molecule 202. An analyte molecule/biosensing molecule 906 in the form of an antibody is attached via an attachment molecule 908 (biotin in this example, avidin may also be used) to the second Dronpa monomer 904 forming a secondary receptor molecule 910. Upon application of 405 nm light 912 the secondary receptor molecule 910 is joined to the base receptor molecule 202 creating an activated bioreceptor molecule, center. Upon application of 488 nm light 914, the secondary receptor molecule 910 disassociates from the base receptor molecule 202 leaving it intact for reuse and/or reconfiguration, right.

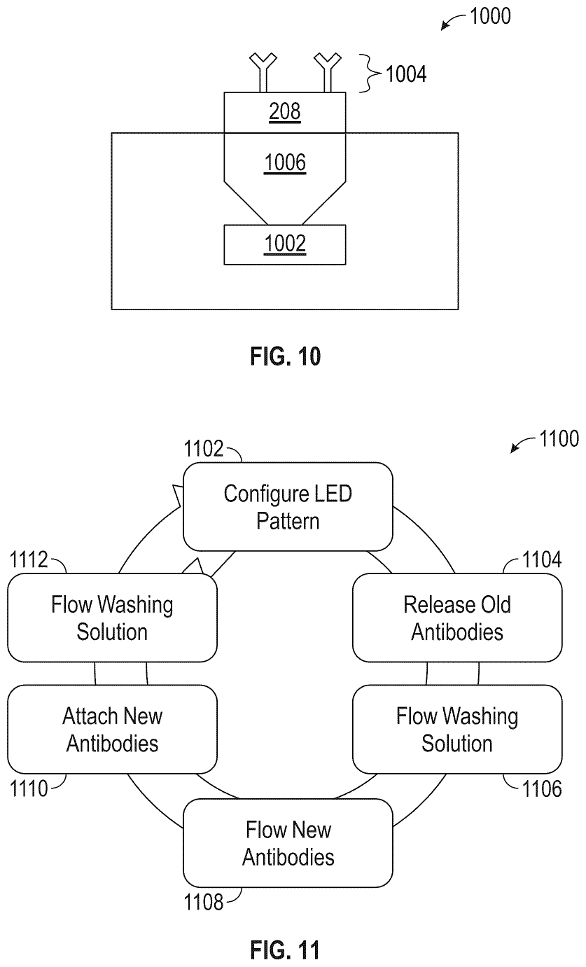

FIG. 10 illustrates in schematic a multi-level sensor platform embodiment 1000. Within the sensor platform stack is a bottom waveguide layer. The bottom waveguide layer 1002 is connected to the same or one or more different light sources 102 than that shown in FIG. 1. The bottom waveguide 1002 is situated below the sensor element 208 (illustrated as a “functionalized waveguide” with base receptor molecules attached to secondary receptor molecules 1004) and is configured to emit light 1006 through the sensor element 208 to affect the above-described photo-sensitive steps.

FIG. 11 illustrates a method 1100 in accordance with embodiments of the disclosure. Starting at the 12 o'clock position and proceeding clockwise: First a lighting pattern, generated by LED emissions, is configured 1102 to interact with the sensor elements of a sensor. Next, the sensor elements of a used sensor are exposed to the LED light pattern releasing the old antibodies 1104 (secondary receptor molecules) from the base receptor molecules. A washing solution is then flowed 1106 over the sensor elements to remove the old secondary receptor molecules. A solution with a known concentration of new antibodies serving as secondary receptor molecules (a “recharging solution”) is then flowed over the sensor elements 1108. The new antibodies are attached 1110 via the LED light pattern. A washing solution is then flowed 1112 over the sensor elements leaving them clean and ready for exposure to the sample environment. The cycle may be repeated with the light pattern focusing on one or more sensor elements to configure each sensor element to detect the same or different targets of interest.

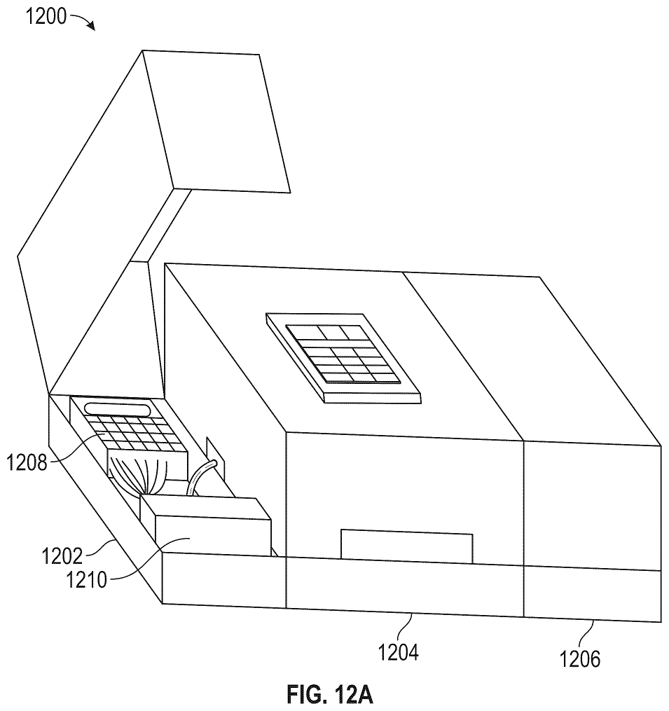

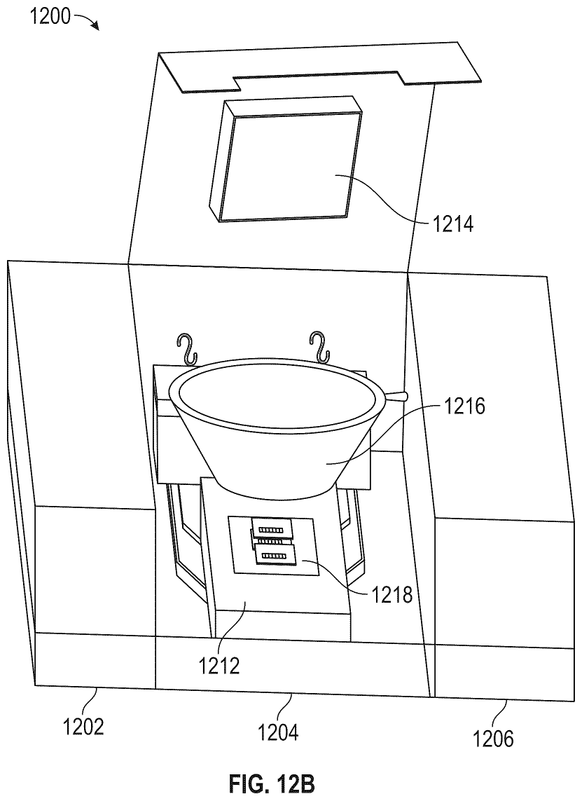

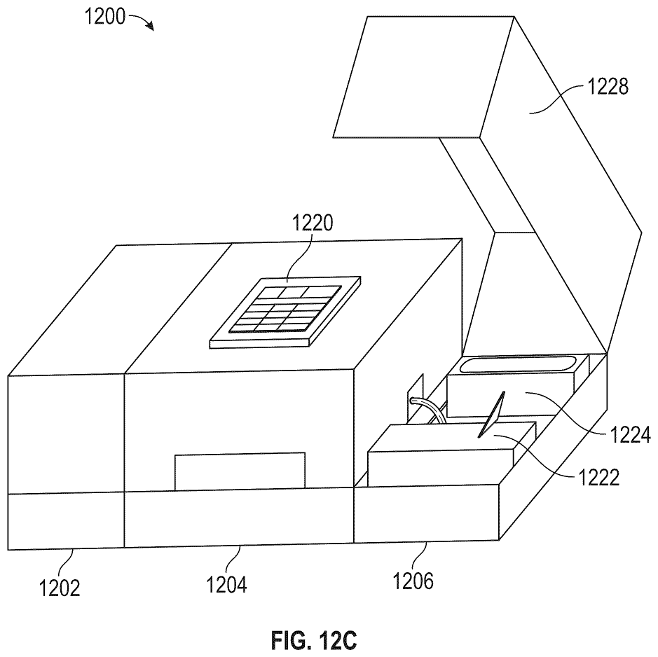

FIG. 12 illustrates in schematic a machine 1200 in accordance with embodiments of the disclosure. The machine is composed of three main sections/areas: a solutions reservoir and fluid handling section 1202 (FIG. 12A), a sensor body and illumination section 1204 (FIG. 12B), and a waste removal and storage area 1206 (FIG. 12C). The solutions reservoir and fluid area 1202 contains one or more vials 1208 of analyte molecules and/or secondary receptor molecules. The area further contains one or more reservoirs of a “washing” solution (part of structure holding vials 1208). Solutions are removed from their respective vials/reservoirs via microfluidic switch and pressure-driven pump 1210 to the sensor body and illumination section 1204. The sensor body and illumination section 1204 is configured to receive one or more sensor chips on a receiving bed configured within a microfluidic chamber 1212. The microfluidic chamber 1212 is configured to receive the fluids from solutions reservoir and fluid area 1202 and apply appropriate solutions to the sensor element(s) of one or more sensor chips. The microfluidic chamber 1212 is kept thermally stable by monitoring temperature via the use of one or more thermocouples. The interior of the upper housing of the sensor body and illumination section 1204 is configured to house one or more light sources 1214; in this case, an LED array that is positioned above a magnification lens 1216. In operation, the LED array alone or in combination with one or more masking devices within the magnification lens 1216 apparatus is configured to illuminate one or more selected areas of the one or more sensor chips placed within the microfluidic chamber 1212 which is further configured with a transparent top 1218. The external surface of the upper housing is equipped with a user interface device such as keypad and display, touchpad, etc 1220. The waste removal and storage area 1206 is equipped with a vacuum pump 1222 and waste well 1224 fluidically coupled to each other and the microfluidic chamber 1212. In operation the vacuum draws waste fluids from the microfluidic chamber 1212 to the waste well 1224 where the waste fluids are subsequently collected and either disposed or recovered to be reused. The waste removal and storage area 1206 is further equipped with a retractable cover 1228 configured to protect the operations conducted in the interior of the machine.

Where elements are presented in list format (e.g., in a Markush group), it is understood that each possible subgroup of the elements is also disclosed, and any one or more elements can be removed from the list or group.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment found within the prior art, can be explicitly excluded from the claims, regardless of whether the specific exclusion is recited in the specification.

Headings are included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated herein by reference in their entirety to the same extent as if each patent literature or non-patent literature were specifically and individually indicated to be incorporated herein by reference in its entirety.

Unless otherwise defined, all technical and scientific terms used herein have the same meaning as commonly understood by one of ordinary skill in the art to which this disclosure belongs.

Where a range of values is provided, it is understood that each intervening value between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both limits, ranges excluding both of those included limits are also included in the invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The term “exemplary” as used herein means “serving as an example, instance or illustration”. Any embodiment or feature characterized herein as “exemplary” is not necessarily to be construed as preferred or advantageous over other embodiments or features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Likewise, in a similar vein, the phrase “and the like” when included as part of a list is intended to convey that while the list is inclusive it is not necessarily exhaustive but those of skill in the art recognizing the guideposts provided in the listing have no problems recognizing specific compounds that fit within the more broadly disclosed classes. The phrase “and the like” explicitly recognizes those molecules and compounds, often too numerous to list, that fit within the disclosed parameters.

It should also be understood that, in certain methods described herein that include more than one step or act, the order of the steps or acts of the method is not necessarily limited to the order in which the steps or acts of the method are recited unless the context indicates otherwise.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

While the present disclosure has been described with reference to an exemplary embodiment or embodiments, it will be understood by those skilled in the art that various changes may be made, and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure is not limited to the particular embodiment disclosed as the best mode contemplated for carrying out this present disclosure, but that the present disclosure will include all embodiments falling within the scope of the claims.

What is claimed is:

1. An apparatus, comprising:
a photonic integrated circuit with at least one element; and,
at least one photo-switchable protein attached to at least one element of the photonic integrated circuit.

2. The apparatus of claim 1, wherein the photonic integrated circuit is combined with an integrated circuit.

3. The apparatus of claim 1 wherein the photonic integrated circuit is the whole or part of a: waveguide, power splitter, optical amplifier, optical modulator, sensor, or biosensor.

4. The apparatus of claim 1 wherein the photo-switchable protein is a dimer and wherein the dimerization is controllable via exposure to at least one light source.

5. The apparatus of claim 4 wherein the photo-switchable protein dimer is Dronpa.

6. The apparatus of claim 5 wherein a glutaraldehyde is attached to a Dronpa monomer.

7. The apparatus of claim 3, wherein the sensor is an evanescent field perturbation sensor, comprising:
at least one sensor element with a surface;
at least a first class of base receptor molecules attached to the surface; and,
at least a first class of secondary receptor molecules;
wherein the secondary receptor molecules are attached or detached from the base receptor molecules in response to photons of a prescribed wavelength.

8. The evanescent field perturbation sensor of claim 7 further comprising:
a light source configured to illuminate at least one sensor element.

9. The apparatus of claim 7 wherein the surface is a sample flow area.

10. The apparatus of claim 7 wherein the base receptor molecules are attached to the surface via silanization.

11. The apparatus of claim 7 further comprising at least a second class of secondary receptor molecules; and, wherein the first class and second class of secondary receptor molecules are configured to interact with different targets.

12. The apparatus of claim 7 further comprising at least a second class of secondary receptor molecules; and, wherein the first class and second class of secondary receptor molecules are configured to interact with different components of the same target.

13. A method, comprising:
providing at least one light source;
providing a solution with analyte molecules;
providing a photonic integrated circuit element with a surface attaching to the surface receptor molecules;
flowing the solution with analyte molecules over the surface of the photonic integrated circuit element;
engaging the light source and binding the analyte molecules to the receptor molecules.

14. The method of claim 13 wherein the receptor molecules are attached via silanization.

15. The method of claim 13 wherein the surface is part of a sample flow area.

16. The method of claim 13 wherein the analyte molecules are at least one selected from the group of: antibodies, antigens, enzymes, nucleic acids, a cell, a cellular structures, and a polymer.

17. The method of claim 13 further comprising:
using the photonic integrated circuit element; and,
recharging the photonic integrated circuit element after use.

18. The method of claim 17 wherein recharging the photonic integrated circuit element reconfigures the element.

19. The method of claim 13 wherein the method is performed with an apparatus according to claim 1.

20. A method of reusing or reconfiguring a photonic integrated circuit, comprising:
providing at least one photonic integrated circuit with at least one used element;
configuring a lighting pattern to interact with the at least one used element;
exposing the at least one used element to light in the lighting pattern, releasing secondary receptor molecules from base receptor molecules;
flowing at least a first washing solution over the at least one used element, removing the released secondary receptor molecules creating a cleaned element;
flowing at least a first recharging solution with new secondary receptor molecules over the cleaned element;
exposing the new secondary receptor molecules and the cleaned element to light in the lighting pattern, binding the new secondary receptor molecules to the base receptor molecules; and,
optionally repeating the above steps for one or more additional elements in series or in parallel.

21. An apparatus configured to perform the method of claim 20.
